(19) United States
(12) Patent Application Publication
(10) Pub. No.: US 2017/0135674 A1
(43) Pub. Date: May 18, 2017

(54) WIRELESS ULTRASONIC PROBE AND ULTRASONIC MACHINE

(71) Applicant: General Electric Company, Schenectady, NY (US)

(72) Inventors: Kevin Wang, Wuxi (CN); Xinchao Wang, Wuxi (CN)

(21) Appl. No.: 15/129,575

(22) PCT Filed: Mar. 2, 2015
(86) PCT No.: PCT/US2015/018225

§ 371 Date: Sep. 27, 2016

(30) Foreign Application Priority Data
Apr. 3, 2014 (CN) 201410133716.9

(51) Int. Cl. A61B 8/00

(52) U.S. Cl. CPC A61B 8/4472; A61B 8/546; A61B 8/56

(57) ABSTRACT

Embodiments of the present invention relate to a wireless ultrasonic probe and an ultrasonic machine. The wireless ultrasonic probe comprises: a probe body for transmitting and receiving ultrasonic waves; and a heat sink comprising a first end portion and a second end portion, wherein at least a part of the second end portion is disposed within the probe body, and the first end portion is formed in a manner that the second end portion extends to the outside of the probe body. The wireless ultrasonic probe can efficiently dissipate heat generated thereby during charging and discharging operation procedures.

### TECHNICAL FIELD

Embodiments of the present invention relate to the technical field of probes, and particularly, to a wireless ultrasonic probe and an ultrasonic machine.

### BACKGROUND ART

An ultrasonic probe, which is mainly used for ultrasonic diagnosis, can convert electrical signals into ultrasonic signals for transmission, and convert ultrasonic echo waves from an object being detected into electrical signals before being transmitted to an ultrasonic main body for subsequent processing.

A wireless ultrasonic probe transmits data between an ultrasonic main body and a probe by the way of wireless transmission. Since plural connecting cables are removed from the ultrasonic machine, it is possible to facilitate an operator in carrying out ultrasonic examination.

However, an existing wireless ultrasonic probe has some difficulty in heat dissipation. On one hand, a transducer control circuit in the ultrasonic probe is sealed within narrow space contained in the probe body; and on the other hand, a charging and discharging circuit of a built-in battery of the wireless ultrasonic probe also generates heat during the operation process.

Therefore, a need exists for a wireless ultrasonic probe and an ultrasonic machine, which can allow excellent heat dissipation on the wireless ultrasonic probe.

### SUMMARY OF THE INVENTION

The object of the present invention is to provide a wireless ultrasonic probe and an ultrasonic machine, which can efficiently dissipate heat generated by the wireless ultrasonic probe during its operation and can charge the wireless ultrasonic probe at low costs.

One embodiment of the present invention provides a wireless ultrasonic probe, comprising: a probe body for transmitting and receiving ultrasonic waves; and a heat sink comprising a first end portion and a second end portion, wherein at least a part of the second end portion is disposed within the probe body, and the first end portion is formed in a manner that the second end portion extends to the outside of the probe body.

Another embodiment of the present invention provides an ultrasonic machine comprising a wireless ultrasonic probe comprising: a probe body for transmitting and receiving ultrasonic waves; and a heat sink comprising a first end portion and a second end portion, wherein at least a part of the second end portion is disposed within the probe body, and the first end portion is formed in a manner that the second end portion extends to the outside of the probe body.

### BRIEF DESCRIPTION OF THE DRAWINGS

In conjunction with the accompanying drawings, the embodiments of the present invention will be described, so that the present invention can be better understood. In the accompanying drawings:

FIG. 1 is a schematic diagram illustrating an overall structure of one embodiment of a wireless ultrasonic probe according to the present invention;

FIG. 2 is a schematic diagram illustrating one embodiment of a heat sink of a wireless ultrasonic probe according to the present invention;

FIG. 3 is a schematic diagram illustrating one embodiment of charging and discharging means of a wireless ultrasonic probe according to the present invention; and

FIG. 4 is a schematic diagram illustrating a discharging operation state of a wireless ultrasonic probe according to an embodiment of the present invention.

### DETAILED DESCRIPTION OF THE EMBODIMENTS

Hereinafter, specific embodiments of the present invention will be described. It is to be noted that, during the process of detailed description of these embodiments, for the sake of concise and clear description, the present description can not possibly describe in detail all features of practical embodiments. It should be understood that, during the process of actual implementation of any embodiment, just as in the process of any engineering or design project, in order to achieve developers' specific purposes and meet system-related or business-related constraints, it is customary to make a variety of specific decisions, which also involves a change from one embodiment to another embodiment or the other way around. In addition, it should also be understood that, although efforts made in such developing process might be complex and lengthy, persons of ordinary skill in the art associated with the disclosure of the present invention would find it nothing but conventional techniques to make modifications in some design, manufacturing, production or the like on the basis of the technical contents disclosed by the present disclosure; and the present disclosure shall not be construed to be insufficient.

Unless defined otherwise, technical terms or scientific terms used in the claims and the description shall carry conventional meanings as construed by persons of ordinary skill in the art which the present invention pertains to. The “first”, “second”, and the like used in the description and the claims of the patent application for an invention do not denote any order, quantity, or importance, but are simply used to make a distinction between different components. The expressions “one”, “a”/“an” or the like do not intend to limit the quantity, but indicate presence of at least one. Such expressions as “comprise”, “include” and the like mean that an element or object present prior to the “comprise” or “include” covers elements or objects listed subsequent to the “comprise” or “include” and their equivalent elements, not excluding other elements or objects. The expression “connect”, “join” or the like is neither limited to physical or mechanical connection, nor limited to direct or indirect connection.

In order to make the object, the technical solution and the advantages of the present invention clearer, the technical solution of the present invention will be clearly and thoroughly described hereinafter in conjunction with specific embodiments of the present invention and the respective drawings. Obviously, the embodiments described are only a part of the embodiments of the present invention, rather than all the embodiments. On the basis of the embodiments of the present invention, other embodiments obtained by a person ordinarily skilled in the art without expending inventive labor all belong to the scope claimed by the present invention.

According to one embodiment of the present invention, a wireless ultrasonic probe is provided.

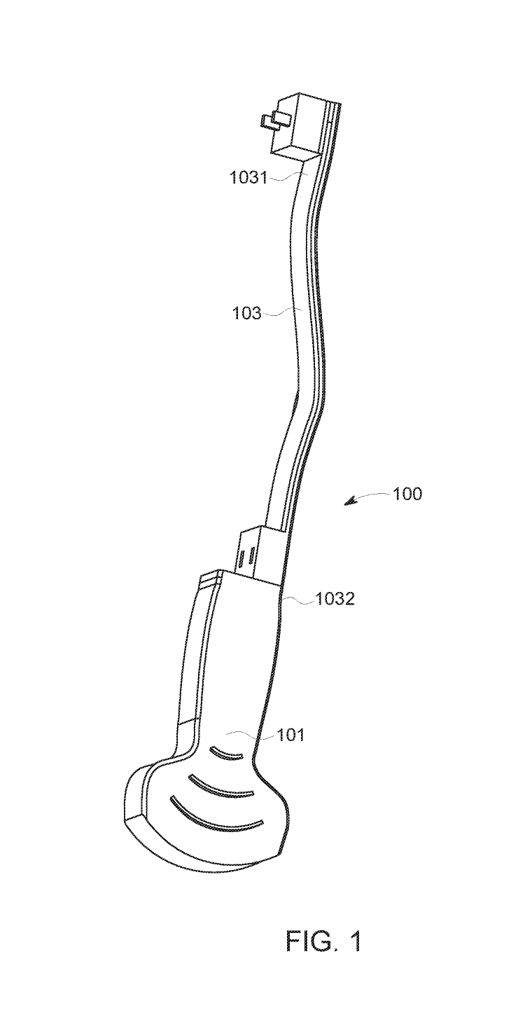

Referring to FIG. 1, which is a schematic diagram illustrating an overall structure of one embodiment of a wireless ultrasonic probe 100 according to the present invention.

The wireless ultrasonic probe 100 may include a probe body 101 and a heat sink 103. The probe body 101, which can be used to transmit and receive ultrasonic waves, can include an ultrasonic transducer and a control circuit for controlling the ultrasonic transducer (not shown in FIG. 1). The heat sink 103 may include a first end portion 1031 and a second end portion 1032. Therein, at least a part of the second end portion 1032 is disposed within the probe body 101, and the first end portion 1031 is formed in a manner that the second end portion 1032 extends to the outside of the probe body. As such, heat generated within the probe body 101 can be dissipated via the heat sink 103.

In one embodiment of the present invention, a heat sink 103 can be of a flexible strip shape and can have a hollow structure, and its hollow portion can be filled with a flexional thermally conductive material. In one embodiment of the present invention, the thermally conductive material can be silicone oil.

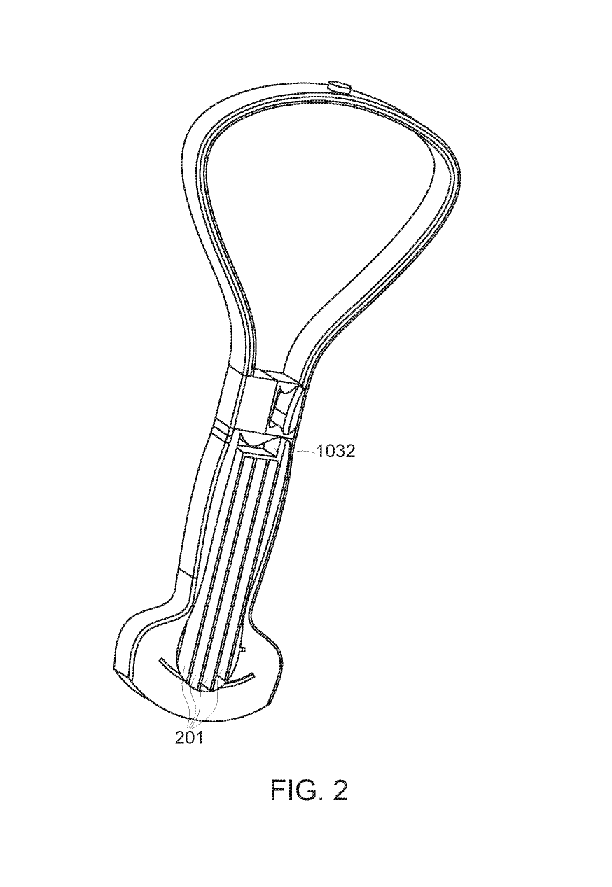

Referring to FIG. 2, which is a schematic diagram illustrating one embodiment of a heat sink of a wireless ultrasonic probe according to the present invention. In order to dissipate heat generated within the probe body in a more efficient manner, in one embodiment of the present invention, thermally conductive patches 201 can be provided on a circuit board and an ultrasonic transducer within the probe body. The thermally conductive patches 201 each can have one face thereof cling against means or locations which generate relatively large amounts of heat, such as a high-voltage pulse generating circuit, an analog front-end circuit and the like. The thermally conductive patches 201 can be connected to a second end portion 1032 of a heat sink 103, so as to dissipate heat generated by electric devices through the heat sink 103.

In one embodiment of the present invention, when a plurality of circuit boards are provided within a probe body and are stacked together in a flexible and rigid bonding manner, a thermally conductive patch can be provided between two adjacent circuit boards and these thermally conductive patches are connected to a second end portion 1032 of a heat sink 103.

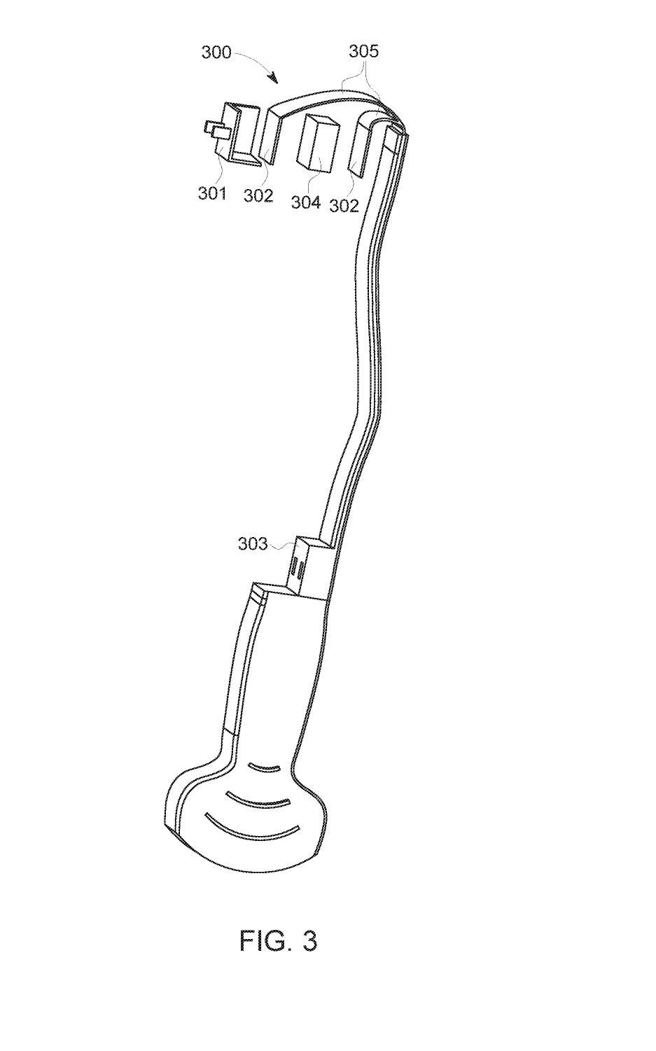

Referring to FIG. 3, which is a schematic diagram illustrating one embodiment of charging and discharging means 300 of a wireless ultrasonic probe according to the present invention.

As shown in FIG. 3, in one embodiment of the present invention, the charging and discharging means 300 can include a charging and discharging plug 301, a charging and discharging circuit 302 and a power supply socket 303. The charging and discharging plug 301, the charging and discharging circuit 302 and a battery 304 of the wireless ultrasonic probe can be disposed at a first end portion 1031 of a heat sink 103. When the charging and discharging plug 301 is plugged into an external power supply, a charging circuit of the wireless ultrasonic probe is on, and electric power of the external power supply charges the battery 304 via the charging and discharging plug 301 and the charging and discharging circuit 302. When the charging and discharging plug 301 is plugged into the power supply socket 303, a power supply circuit of the wireless ultrasonic probe is on, and electric energy stored in the battery 304 is supplied to electric devices within the probe body via the charging and discharging circuit 302. In one embodiment of the present invention, the power supply socket 303 can be disposed on the probe body, i.e., is designed integral with the probe body 101, and as such, when the charging and discharging plug 301 is plugged into the power supply socket 303, a heat sink 103 can be bent to have an annular shape, so as to facilitate the user's operation.

Since the charging and discharging circuit 302 will generate heat during the charging and discharging procedures, in order to carry out heat dissipation on the charging and discharging circuit 302, in one embodiment of the present invention, thermally conductive patches 305 can be provided at locations adjacent to the charging and discharging circuit 302. One face of each of the thermally conductive patches 305 can be in contact with areas or components which generate relatively large amounts of heat in the charging and discharging circuit 302. The thermally conductive patches 305 can dissipate heat generated by the charging and discharging circuit 302 via the heat sink 103 by way of connection to the first end portion 1031 of the heat sink 103.

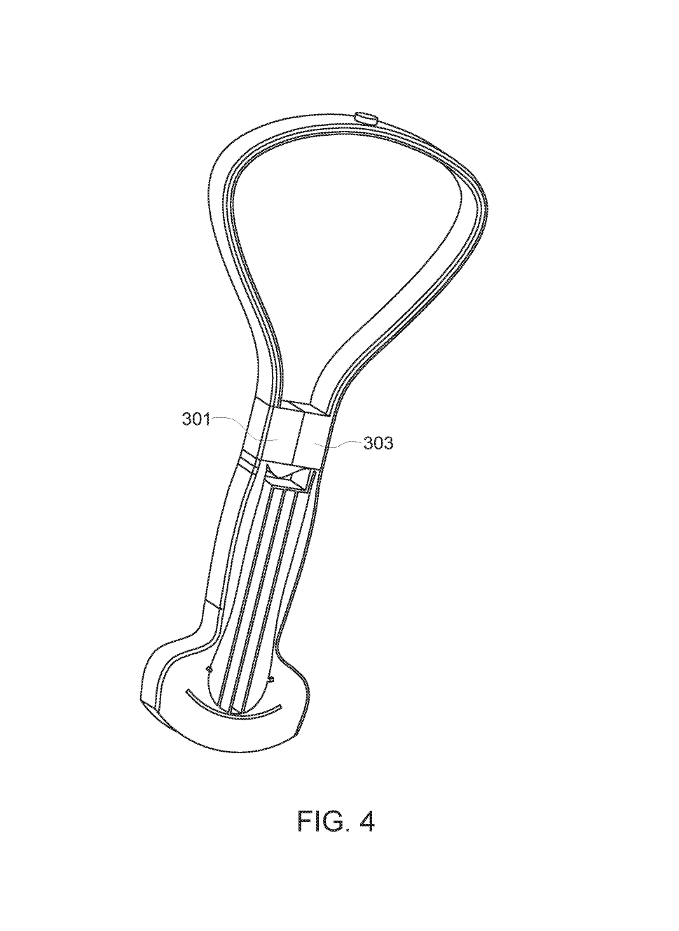

Referring to FIG. 4, which is a schematic diagram illustrating a discharging operation state of a wireless ultrasonic probe according to an embodiment of the present invention. When it is necessary to use the wireless ultrasonic probe of an embodiment of the present invention to carry out ultrasonic examination, a charging and discharging plug 301 is only required to be plugged into a power supply socket 303, such that it is possible to supply power to electric devices within the probe via the power supply socket 303.

The wireless ultrasonic probe according to the embodiments of the present invention has been described thus far. The wireless ultrasonic probe is designed to have a heat sink extending out of the probe body, and a charging and discharging circuit is designed at one end of the heat sink. In this way, it is possible to efficiently dissipate heat generated by the wireless ultrasonic probe during the charging and discharging operation procedures and charge the wireless ultrasonic probe at low costs.

The foregoing description is nothing but the embodiments of the present invention and is not intended to limit the present invention. For one skilled in the art, the present invention can undergo various modifications and variations. Any modification, equivalent replacement, improvement and the like made within the spirit and principles of the present invention shall be included in the scope claimed by the present invention.

1. A wireless ultrasonic probe, characterized by comprising:
a probe body for transmitting and receiving ultrasonic waves; and
a heat sink comprising a first end portion and a second end portion, wherein at least a part of the second end portion is disposed within the probe body, and the first end portion is formed in a manner that the second end portion extends to the outside of the probe body.

2. The wireless ultrasonic probe according to claim 1, characterized in that, the probe body further comprises:
a first thermally conductive patch, one end of the first thermally conductive patch being in contact with a heat generating member within the probe body and the other end thereof being connected to the second end portion.

3. The wireless ultrasonic probe according to claim 1, characterized in that, the heat sink has a flexible strip shape.

4. The wireless ultrasonic probe according to claim 1, characterized in that, the heat sink has a hollow structure.

5. The wireless ultrasonic probe according to claim 4, characterized in that, the heat sink has its hollow portion filled with a flexional thermally conductive material.

6. The wireless ultrasonic probe according to claim 1, characterized in that, the wireless ultrasonic probe further comprises:
charging and discharging means for charging a battery of the wireless ultrasonic probe or supplying power to electric devices within the probe body.

7. The wireless ultrasonic probe according to claim 6, characterized in that, the charging and discharging means further comprises:
a power supply socket, disposed on the probe body for receiving electric power supplied by the charging and discharging means to the electric devices within the probe body;
a charging and discharging plug, wherein said charging and discharging plug, when plugged into an external power supply, receives electric power from the external power supply, and when plugged into the power supply socket, transmits electric power to the electric devices within the probe body; and
a charging and discharging circuit, wherein the charging and discharging circuit charges the battery when the charging and discharging plug is plugged into the external power supply, and supplies power to the electric devices within the probe body when the charging and discharging plug is plugged into the power supply socket.

8. The wireless ultrasonic probe according to claim 7, characterized in that, the charging and discharging plug and the charging and discharging circuit are disposed at said first end portion.

9. The wireless ultrasonic probe according to claim 8, characterized in that, the charging and discharging means further comprises:
a second thermally conductive patch, one end of the second thermally conductive patch being in contact with a heat generating member within the charging and discharging means and the other end thereof being connected to the first end portion.

10. An ultrasonic machine, characterized by comprising a wireless ultrasonic probe according to claim 1.
